(19) United States
(12) Patent Application Publication
(10) Pub. No.: US 2026/0035510 A1
(43) Pub. Date: Feb. 5, 2026

(54) ETHYLENE GLYCOL COMPOSITION, PREPARATION METHOD THEREOF, AND POLYESTER PREPARED FROM THE ETHYLENE GLYCOL COMPOSITION

(71) Applicants: Guoping CHEN, Yulin (CN); HIGHCHEM CO., LTD, Tokyo (JP)

(72) Inventors: Guoping CHEN, Yulin (CN); Yuichi TAKA, Tokyo (JP); Qipeng DAI, Tokyo (JP); Changjiang WU, Tokyo (JP); Changlin LI, Tokyo (JP); Shiriri JO, Tokyo (JP)

(73) Assignees: Guoping CHEN, Yulin (CN); HIGHCHEM CO., LTD, Tokyo (JP)

(21) Appl. No.: 18/995,870

(22) PCT Filed: Aug. 10, 2023
(86) PCT No.: PCT/CN2023/112280

§ 371 Date: Jan. 17, 2025

(30) Foreign Application Priority Data
Aug. 12, 2022 (CN) 202210969655.4

(51) Int. Cl. C08G 63/183; C07C 29/36

(52) U.S. Cl. CPC C08G 63/183; C07C 29/36

(57) ABSTRACT

An ethylene glycol composition, a preparation method thereof, and a polyester prepared from the ethylene glycol composition are provided. The ethylene glycol composition of the present invention has a specific content of propylene glycol, butanediol and hexanediol. The polyester obtained by polymerizing the ethylene glycol composition with a dibasic acid such as terephthalic acid has a low glass transition temperature and excellent toughness while maintaining excellent strength and heat resistance, which can greatly broaden the application scope of the polyester, and is particularly useful for low-temperature dyeing fibers.

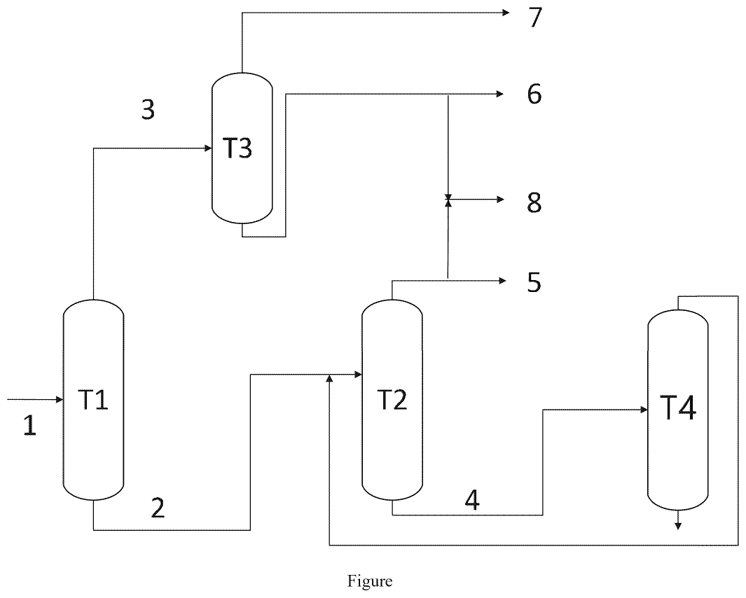

### CROSS REFERENCE TO THE RELATED APPLICATIONS

This application is the national phase entry of International Application No. PCT/CN2023/112280, filed on Aug. 10, 2023, which is based upon and claims priority to Chinese Patent Application No. 202210969655.4, filed on Aug. 12, 2022, the entire contents of which are incorporated herein by reference.

### TECHNICAL FIELD

The present invention relates to an ethylene glycol composition, a preparation method thereof, and a polyester prepared from the ethylene glycol composition.

### BACKGROUND

Ethylene glycol is a colorless, odorless, low-toxic, simplest aliphatic diol. It is also an important basic raw material and chemical intermediate, and the most consumed polyol in the world. Ethylene glycol is mainly used in the production of polyester fibers, antifreezes, unsaturated polyester resins, lubricants, plasticizers, non-ionic surfactants and explosives, and the like. In addition, ethylene glycol can also be used in the field such as coatings, photographic developers, brake fluids, inks, and the production of special solvents such as glycol ethers.

Due to its unique properties and huge commercial application value, people have explored a variety of chemical technologies to synthesize ethylene glycol over the years. There are two main synthesis technologies: petroleum method and coal-based synthesis gas method. Under the energy pattern of “lack of oil, little gas, and relatively rich in coal” in my country, the coal-based synthesis gas method, as a new coal chemical process, has attracted much attention for its low cost, low energy consumption, low water consumption and low emissions. However, due to the different reaction mechanisms, process routes, product index and impurity components of the method of producing ethylene glycol from coal-based synthesis gas, the quality of ethylene glycol products is unstable despite for different batches of products produced by the same enterprise, resulting in the quality and yield of ethylene glycol being one of the most difficulties to overcome in many technologies. In addition, as far as ethylene glycol used as a polyester raw material is concerned, there are very strict requirements on the quality of the product, especially the ultraviolet transmittance (hereinafter referred to as “UV value”) index has a great influence on the coloring of downstream polyester fibers, fiber strength and fiber color. Therefore, in order to improve the product quality of ethylene glycol and make the product meet the requirements of “polyester-grade ethylene glycol”, many ethylene glycol manufacturers need to use several distillation towers to purify and refine crude ethylene glycol to remove various organic impurities in ethylene glycol products. When ethylene glycol with unqualified UV transmittance is used in PET polyester, it will affect the quality in terms of gloss, chroma, coloring, etc.

In order to improve the product quality of ethylene glycol, many attempts have been made.

CN112538001A discloses a separation and purification process for coal-based ethylene glycol products and by-products, which refines crude coal-based ethylene glycol to obtain ethylene glycol products through pre-separation, methanol removal, dehydration, ethanol removal, ethanol recovery, ethylene glycol recovery, hydrogenation, ethylene glycol refining and other techniques.

Yuan WANG et al. (Chemical Enterprise Management, 2021, (12), 171-173) found in a method for improving product quality by acidity control in an ethylene glycol distillation system that adjusting the bed temperature and reactor pressure difference in the ethylene glycol synthesis section is the best method for regulating the distillation system.

In addition, CN105585446A discloses an ethylene glycol composition, in particular an ethylene glycol composition obtained by hydrogenation of oxalate.

However, the above methods are improvements made to completely remove impurities such as acids, conjugated aldehydes, and other diols from ethylene glycol products to achieve high-purity polyester-grade ethylene glycol products. No further research has been conducted on the various components in ethylene glycol products to enrich the categories and uses of ethylene glycol products.

### SUMMARY

In view of the above-mentioned situation of the prior art, the inventors have conducted extensive and in-depth research on the quality of ethylene glycol products used as polyester raw materials and ethylene glycol distillation process in the coal-based synthesis gas method, and discovered a new ethylene glycol composition, which, when used as a raw material for polyesters such as polyethylene terephthalate (PET), obtains a modified polyester with new properties. In particular, the polyester obtained by polymerizing the ethylene glycol composition of the present invention with a dibasic acid such as terephthalic acid has a low glass transition temperature and excellent toughness while maintaining excellent strength and heat resistance, which can greatly broaden the scope of use of polyester. In particular, the fiber prepared from the obtained polyester has excellent low-temperature dyeability.

In addition, an improved ethylene glycol product distillation method has been discovered, which can easily obtain the ethylene glycol composition of the present invention. This method not only reduces the energy consumption of the process, but also greatly expands the application field of industrial-grade ethylene glycol products.

One aspect of the present invention relates to an ethylene glycol composition including, preferably consisting of:
(1) at least 99.0% ethylene glycol,
(2) 110-5000 ppm, preferably 150-4000 ppm, more preferably 160-3500 ppm, most preferably 170-3000 ppm propylene glycol,
(3) 110-10000 ppm, preferably 300-9000 ppm, more preferably 800-8000 ppm, most preferably 1000-7000 ppm butanediol, and
(4) 110-10000 ppm, preferably 150-5000 ppm, more preferably 200-3000 ppm, most preferably 250-2000 ppm hexanediol,
in each case based on the total weight of the ethylene glycol composition.

In the ethylene glycol composition of the present invention, the propylene glycol may be 1,2-propylene glycol, 1,3-propylene glycol or 2-methyl-1,3-propylene glycol, preferably 1,2-propylene glycol or 2-methyl-1,3-propylene glycol, and particularly preferably 2-methyl-1,3-propylene glycol. The amount of propylene glycol may be 110-5000 ppm, preferably 150-4000 ppm, more preferably 160-3500 ppm, most preferably 170-3000 ppm, for example, 2000 ppm, 1500 ppm, 1000 ppm or 800 ppm.

In the ethylene glycol composition of the present invention, butanediol may be 1,2-butanediol, 1,3-butanediol or 1,4-butanediol, preferably 1,2-butanediol or 1,3-butanediol, and more preferably 1,2-butanediol. The amount of butanediol may be 110-10000 ppm, preferably 300-9000 ppm, more preferably 800-8000 ppm, and most preferably 1000-7000 ppm, for example 6000 ppm, 5000 ppm, 4000 ppm, 3000 ppm or 2000 ppm.

In the ethylene glycol composition of the present invention, hexanediol may be 1,2-hexanediol, 1,3-hexanediol, 1,4-hexanediol, 1,5-hexanediol or 1,6-hexanediol, preferably 1,2-hexanediol, 1,3-hexanediol, 1,4-hexanediol or 1,5-hexanediol. The amount of hexanediol may be 110-10000 ppm, preferably 150-5000 ppm, more preferably 200-3000 ppm, most preferably 250-2000 ppm, for example 1500 ppm, 1000 ppm or 800 ppm.

In a preferred embodiment, the present invention relates to an ethylene glycol composition including, preferably consisting of:
(1) at least 99.0% ethylene glycol,
(2) 110-5000 ppm, more preferably 150-4000 ppm, more preferably 160-3500 ppm, most preferably 170-3000 ppm 1,2-propylene glycol or 2-methyl-1,3-propylene glycol, preferably 2-methyl-1,3-propylene glycol,
(3) 110-10000 ppm, preferably 300-9000 ppm, more preferably 800-8000 ppm, most preferably 1000-7000 ppm 1,2-butanediol or 1,3-butanediol, and
(4) 110-10000 ppm, preferably 150-5000 ppm, more preferably 200-3000 ppm, most preferably 250-2000 ppm 1,2-hexanediol, 1,3-hexanediol, 1,4-hexanediol or 1,5-hexanediol, in each case based on the total weight of the ethylene glycol composition.

In another preferred embodiment, the present invention relates to an ethylene glycol composition including, preferably consisting of:
(1) at least 99.0% ethylene glycol,
(2) 150-4000 ppm 1,2-propylene glycol or 2-methyl-1,3-propylene glycol, preferably 2-methyl-1,3-propylene glycol,
(3) 300-9000 ppm 1,2-butanediol or 1,3-butanediol, and
(4) 150-5000 ppm 1,2-hexanediol, 1,3-hexanediol, 1,4-hexanediol or 1,5-hexanediol, in each case based on the total weight of the ethylene glycol composition.

In another preferred embodiment, the present invention relates to an ethylene glycol composition including, preferably consisting of:
(1) at least 99.0% ethylene glycol,
(2) 160-3500 ppm 1,2-propylene glycol or 2-methyl-1,3-propylene glycol, preferably 2-methyl-1,3-propylene glycol,
(3) 800-8000 ppm 1,2-butanediol or 1,3-butanediol, and
(4) 200-3000 ppm 1,2-hexanediol, 1,3-hexanediol, 1,4-hexanediol or 1,5-hexanediol, in each case based on the total weight of the ethylene glycol composition.

In another preferred embodiment, the present invention relates to an ethylene glycol composition including, preferably consisting of:
(1) at least 99.0% ethylene glycol,
(2) 170-3000 ppm 1,2-propylene glycol or 2-methyl-1,3-propylene glycol, preferably 2-methyl-1,3-propylene glycol,
(3) 1000-7000 ppm 1,2-butanediol or 1,3-butanediol, and
(4) 250-2000 ppm 1,2-hexanediol, 1,3-hexanediol, 1,4-hexanediol or 1,5-hexanediol, in each case based on the total weight of the ethylene glycol composition.

In another preferred embodiment, the present invention relates to an ethylene glycol composition consisting of:
(1) at least 99.0% ethylene glycol,
(2) 170-3000 ppm 2-methyl-1,3-propanediol,
(3) 1000-7000 ppm 1,2-butanediol or 1,3-butanediol, and
(4) 250-2000 ppm 1,2-hexanediol, 1,3-hexanediol, 1,4-hexanediol or 1,5-hexanediol, in each case based on the total weight of the ethylene glycol composition.

Surprisingly, it is found that since the ethylene glycol composition of the present invention has a specific content of propylene glycol, butanediol and hexanediol, especially 1,2-propylene glycol or 2-methyl-1,3-propylene glycol, and 1,2-butanediol or 1,3-butanediol, as well as 1,2-hexanediol, 1,3-hexanediol, 1,4-hexanediol or 1,5-hexanediol, the polyester obtained by polymerizing the ethylene glycol composition with a dibasic acid such as terephthalic acid has a low glass transition temperature and excellent toughness while maintaining excellent strength and heat resistance, thereby greatly broadening the application scope of the polyester.

Another aspect of the present invention relates to a method for preparing an ethylene glycol composition, including the steps of:
(1) reacting CO with methanol to generate dimethyl oxalate, then hydrogenating dimethyl oxalate to generate ethylene glycol, and passing the ethylene glycol through a methanol recovery tower and a dehydration tower to obtain crude ethylene glycol;
(2) feeding the crude ethylene glycol to a light fraction removal tower, passing a heavy fraction of the light fraction removal tower to an ethylene glycol product tower, and distilling the heavy fraction in the ethylene glycol product tower to obtain an ethylene glycol product;
(3) feeding a light fraction of the light fraction removal tower to a specific alcohol recovery tower, distilling the light fraction therein, and obtaining an ethylene glycol solution containing a high concentration of specific alcohol from the bottom of the tower;
(4) mixing the ethylene glycol solution containing a high concentration of specific alcohol obtained in step (3) with the ethylene glycol product obtained in step (2) in appropriate amounts according to the desired concentration of the ethylene glycol composition.

In the context of the present invention, the term “specific alcohol” means the propylene glycol, butanediol and hexanediol as mentioned above, for example 1,2-propylene glycol, 1,3-propylene glycol, 2-methyl-1,3-propylene glycol, 1,2-butanediol, 1,3-butanediol, 1,4-butanediol, 1,2-hexanediol, 1,3-hexanediol, 1,4-hexanediol, 1,5-hexanediol and/or 1,6-hexanediol.

The crude ethylene glycol in step (1) of the present invention is obtained by reacting CO and methanol to generate dimethyl oxalate under the action of a catalyst, and then distilling and purifying, followed by making the dimethyl oxalate to generate crude ethylene glycol under the action of a hydrogenation catalyst. The method of generating ethylene glycol from CO and methanol via dimethyl oxalate is known in the art, for example, see CN105585446A. The general ethylene glycol process uses coal as a raw material, and obtains raw gas CO and H₂ respectively through air separation, gasification, transformation, cleansing and separation and purification. They react with methanol in a methyl nitrite regeneration tower to generate methyl nitrite, which then reacts with CO to generate dimethyl oxalate (DMO). In a hydrogenation reactor, DMO reacts with hydrogen to generate ethylene glycol and methanol.

The top temperature of the methanol recovery tower in step (1) of the method of the present invention is 40-70° C., the bottom temperature is 80-180° C., and the operation is carried out under normal pressure or reduced pressure; the top temperature of the dehydration tower is 40-70° C., the bottom temperature is 80-180° C., and the operation is carried out under normal pressure or reduced pressure.

The top temperature of the light fraction removal tower in step (2) of the method of the present invention is 40-60° C., the bottom temperature is 70-160° C., and the operation is carried out under normal pressure or reduced pressure.

The top temperature of the ethylene glycol product tower in step (2) of the method of the present invention is 100-150° C., and the bottom temperature is 130-230° C.

The theoretical number of plates of the specific alcohol recovery tower in step (3) of the method of the present invention is 5-100, the top temperature is 80-300° C., the bottom temperature is 90-290° C., and the operation is carried out under normal pressure or reduced pressure; the reflux ratio of the light component at the top of the ethylene glycol product tower is 50-120 or full reflux. The “ethylene glycol solution containing a high concentration of specific alcohol” refers to ethylene glycol rich in propylene glycol, butanediol and hexanediol, for example, ethylene glycol rich in 1,2-propylene glycol, 1,3-propylene glycol, 2-methyl-1,3-propylene glycol, 1,2-butanediol, 1,3-butanediol, 1,4-butanediol, 1,2-hexanediol, 1,3-hexanediol, 1,4-hexanediol, 1,5-hexanediol and/or 1,6-hexanediol. In the context of the present invention, “rich” means that the concentration of propylene glycol, butanediol and hexanediol in the ethylene glycol solution containing a high concentration of specific alcohol is higher than that of the ethylene glycol product. Preferably, the concentrations of propylene glycol, butanediol and hexanediol in the ethylene glycol solution containing a high concentration of specific alcohol are 0.1-15 wt %, 0.1-40 wt % and 0.1-10 wt %, respectively.

In step (4) of the present invention, according to the desired concentration of the ethylene glycol composition, the ethylene glycol solution containing a high concentration of specific alcohol obtained in step (3) and the ethylene glycol product obtained in step (2) are mixed in appropriate amounts. In the context of the present invention, “appropriate amount” means mixing the ethylene glycol solution containing a high concentration of specific alcohol obtained in step (3) with the ethylene glycol product obtained in step (2), thereby obtaining the ethylene glycol composition of the present invention having a specific content of ethylene glycol, propylene glycol, butanediol and hexanediol. Therefore, in step (4) of the method according to the present invention, “desired concentration” means the concentration of the specific ethylene glycol, propylene glycol, butanediol and hexanediol as described above for the ethylene glycol composition of the present invention. Preferably, the mixing weight ratio of the ethylene glycol solution containing a high concentration of specific alcohol obtained in step (3) to the ethylene glycol product obtained in step (2) is 1:30-1:500.

The method of the present invention can easily obtain the ethylene glycol composition of the present invention described above. In the coal-based synthesis gas method of the prior art, the crude ethylene glycol obtained is passed through a methanol recovery tower, a dehydration tower, a light fraction removal tower, an ethylene glycol product tower, an ethylene glycol recovery tower and an ethylene glycol concentration tower to obtain a polyester-grade ethylene glycol product. Different from this, in the present invention, the process of the light fraction removal tower is improved, and the light fraction of the light fraction removal tower is further separated to obtain ethylene glycol containing a specific alcohol. The ethylene glycol contains a high concentration of specific alcohol, and can be appropriately mixed with the ethylene glycol product of the ethylene glycol product tower to achieve the purpose of adjusting the concentration of the final ethylene glycol composition.

The ethylene glycol composition of the present invention has a specific content of propylene glycol, butanediol and hexanediol. Therefore, the polyester obtained by polymerizing the ethylene glycol composition with a dibasic acid such as terephthalic acid has a low glass transition temperature and excellent toughness while maintaining excellent strength and heat resistance, which can greatly broaden the application scope of polyester. In particular, the polyester of the present invention is applicable for producing fibers that can be dyed at low temperatures.

Thus, another aspect of the present invention relates to a polyester including a polymerized monomer unit derived from the ethylene glycol composition of the present invention.

In addition, the polyester of the present invention also contains a polymerized monomer unit derived from an aromatic dicarboxylic acid and its derivatives. Preferred aromatic dicarboxylic acid and its derivatives are selected from terephthalic acid, phthalic acid, isophthalic acid, naphthalene dicarboxylic acid and anhydrides thereof, preferably terephthalic acid and/or its anhydrides.

Optionally, the polyester of the present invention may contain a polymerized monomer unit derived from an aliphatic dicarboxylic acid and its derivatives. Suitable aliphatic dicarboxylic acid and its derivatives are selected from succinic acid, adipic acid, suberic acid, or their anhydrides. If present, the amount of aliphatic dicarboxylic acid and derivatives thereof is 0-30 wt %, preferably 0-10 wt %, more preferably 0-5 wt %, based on the total moles of all polymerized dicarboxylic acid monomer units. Preferably, the polyester of the present invention does not contain an aliphatic dicarboxylic acid and derivatives thereof.

Optionally, the polyester of the present invention may contain a small amount of polymerized monomer unit derived from polyacids and their derivatives. The polyacids and their derivatives may be selected from trimellitic acid, pyromellitic acid or their anhydrides. If present, the amount of polybasic organic acid and its derivatives is 0-2 wt %, preferably 0-0.5 wt %, based on the total moles of all polymerized monomer units. Preferably, the polyester of the present invention does not contain a polybasic organic acid and derivatives thereof.

The polyester of the present invention can be obtained by polycondensing the ethylene glycol composition of the present invention with a dibasic acid or a polyacid monomer, preferably by polycondensing the ethylene glycol composition of the present invention with terephthalic acid.

The polycondensation reaction is known in the art, including: putting all monomers, catalysts and stabilizers into a reaction vessel, and after excluding air, carrying out an esterification reaction; when the amount of esterification distillate reaches 75-85% of the theoretical value, the esterification reaction is terminated; and then the polycondensation reaction is carried out.

The temperature of the esterification reaction is 180-260° C. and the pressure is 0.2-0.4 MPa. The temperature of the polycondensation reaction is 220-290° C., the pressure is 10-60 Pa, and the duration is 0.5-10 hours, preferably 2-8 hours.

The catalyst used is known in the art, for example, one or more of stannous octoate, stannous isooctanoate, stannous oxalate, stannous chloride, stannous oxide, antimony glycolate, antimony trioxide, antimony acetate, tetrabutyl titanate, tetrapropyl titanate, titanium oxalate, titanium acetate and titanium tetrachloride.

The stabilizer used is known in the art, for example phosphoric acid, alkyl phosphate, triphenyl phosphate, alkyl diaryl phosphate or mixed alkyl aryl phosphate.

The polyester of the present invention has a glass transition temperature below 60° C. and an intrinsic viscosity greater than 0.60 g/dL.

It is surprisingly found that the fiber produced from the polyester of the present invention has low temperature dyeability and good color fastness.

The ethylene glycol composition of the present invention is useful in the production of polyester resins, antifreeze solutions or unsaturated resins.

Therefore, the present invention also relates to the use of the ethylene glycol composition of the present invention in the production of polyester resins, antifreeze solutions or unsaturated resins.

### BRIEF DESCRIPTION OF THE DRAWINGS

FIGURE is a process flow chart for preparing the ethylene glycol composition of the present invention, wherein:

T1 is a light fraction removal tower, T2 is an ethylene glycol product tower, T3 is a specific alcohol recovery tower, and T4 is an ethylene glycol recovery tower. 1 is a crude ethylene glycol feed, 2 is a heavy fraction at the bottom of the light fraction removal tower, 3 is a light fraction at the top of the light fraction removal tower, 4 is a heavy fraction at the bottom of the ethylene glycol product tower, 5 is an ethylene glycol product, 6 is an ethylene glycol solution containing a high concentration of specific alcohol, 7 is a light component such as dimethyl oxalate and methyl glycolate, and 8 is a final ethylene glycol composition.

The method of the present invention is described in detail below with reference to the FIGURE.

CO and H₂ react with methanol in a methyl nitrite regeneration tower to generate methyl nitrite, which then reacts with CO to generate dimethyl oxalate (DMO). In a hydrogenation reactor, DMO reacts with hydrogen to generate ethylene glycol and methanol, which are then passed through a methanol recovery tower and a dehydration tower to obtain a crude ethylene glycol feed 1. Feed 1 is a mixed ethylene glycol solution containing dimethyl oxalate, methyl glycolate, ethylene glycol monomethyl ether, propylene glycol, butanediol, pentanediol, hexanediol, ethylene carbonate, diethylene glycol and other substances, which is passed into the light fraction removal tower T1, and the heavy fraction 2 at the bottom of the tower after distillation and separation is sent to the ethylene glycol product tower T2. After distillation in the ethylene glycol product tower T2, an ethylene glycol product 5 that meets national standards is obtained, and the heavy fraction 4 at the bottom of the tower is sent to the ethylene glycol recovery tower T4, and the top component of T4 is recycled back to the ethylene glycol product tower T2 for recycling and reuse. The light fraction 3 at the top of the light fraction removal tower is sent to the specific alcohol recovery tower T3 to separate the required alcohols. The light component 7 containing dimethyl oxalate, methyl glycolate, etc. is treated as waste liquid, and the ethylene glycol solution 6 containing a high concentration of specific alcohol is obtained from the bottom of the tower. Depending on the working conditions, 6 can be directly sold as a special specification product, or 6 can be mixed with 5 in appropriate amounts to obtain a final ethylene glycol composition 8 containing a specific concentration of alcohol.

### DETAILED DESCRIPTION OF THE EMBODIMENTS

The present invention is further described below by way of examples, but the present invention is not limited thereby.

### Testing Methods

\textcircled{1} Intrinsic viscosity test: Referring to the capillary viscometer method in GB/T14190-2017 fiber-grade polyester chips (PET) experimental method, the intrinsic viscosity [η] of the polyester is measured.
\textcircled{2} Thermal performance test: Using DSC, the polyester sample is first heated to 110° C., stabilized for 10 minutes, and then cooled to 0° C., then heated to 280° C. at 10° C./min, stabilized for 10 minutes, and finally cooled to 0° C. at 10° C./min to test the glass transition temperature Tg and melting point Tm of the polyester.
\textcircled{3} Strength and toughness test: Referring to the test of tensile properties of plastics in GB/T1040.2-2006, the tensile strength and elongation at break of the polyesters of the examples and comparative examples are tested.

### Example 1

Feed 1 is a mixed ethylene glycol solution containing dimethyl oxalate, methyl glycolate, ethylene glycol monomethyl ether, propylene glycol, butanediol, pentanediol, hexanediol, ethylene carbonate, diethylene glycol and other substances, which is passed into the light fraction removal tower T1, with a tower top temperature of 50° C., a tower bottom temperature of 140° C., and normal pressure operation. After distillation and separation, the heavy fraction 2 at the bottom of the tower is sent to the ethylene glycol product tower T2. After distillation in the ethylene glycol product tower T2 at a tower top temperature of 120° C., a tower bottom temperature of 180° C. and normal pressure, an ethylene glycol product 5 that meets national standards is obtained, and the heavy fraction 4 at the bottom of the tower is sent to the ethylene glycol recovery tower T4, and the top component of T4 is recycled back to the ethylene glycol product tower T2 for recycling and reuse. The light fraction at the top of the light fraction removal tower T1 is sent to the specific alcohol recovery tower T3, and the light component 7 containing dimethyl oxalate, methyl glycolate, etc. is treated as waste liquid, and the ethylene glycol solution 6 containing a high concentration of specific alcohol is obtained from the bottom of the tower. The operating conditions of the specific alcohol recovery tower are: theoretical plate number 23, tower top temperature 223° C., tower bottom temperature 254° C., and normal pressure operation. 6 and 5 are mixed at a weight mixing ratio of 1:150 to obtain a final ethylene glycol composition 8. Data of the obtained ethyleneglycol composition is shown in Table 1.

TABLE 1
Ethylene glycol composition table

| | 6 | | | |
|---|---|---|---|---|
| | Ethylene glycol | | 8 | |
| | solution containing a | | Final | |
| | high concentration | | ethylene glycol | |
| Content | of specific alcohol | | composition | |
| ethylene glycol | 61 | wt % | 99.6 | wt % |
| 1,2-propanediol + | 9.65 | wt % | 639 | ppm |
| 2-methyl-1,3-propanediol | | | | |
| 1,2-butanediol | 27.4 | wt % | 1825 | ppm |
| 1,2-, 1,3-, 1,4-, 1,5-hexanediol | 2.1 | wt % | 437 | ppm |

### Comparative Example 1

Comparative Example 1 is carried out in the same manner as Example 1, except that there is no specific alcohol recovery tower, and therefore there is no step of mixing the ethylene glycol solution containing a high concentration of specific alcohol in the specific alcohol recovery tower with the ethylene glycol product. Data of the obtained ethylene glycol composition is shown in Table 2.

TABLE 2
Ethylene glycol composition table

| | 6 | | |
|---|---|---|---|
| | Ethylene glycol | 8 | |
| | solution containing a | Final | |
| | high concentration | ethylene glycol | |
| Content | of specific alcohol | composition | |
| ethylene glycol | — | 99.9 | wt % |
| 1,2-propanediol | — | 50 | ppm |
| 1,2-butanediol | — | 10 | ppm |
| 1,2-, 1,3-, 1,4-, 1,5-hexanediol | — | 300 | ppm |

### Example 3

Example 1 is repeated, except that the distillation conditions of the specific alcohol recovery tower are changed. The specific distillation conditions are as follows: theoretical plate number 70, tower top temperature 215° C., tower bottom temperature 250° C., and normal pressure operation. 6 and 5 are mixed at a weight mixing ratio of 1:150. Data of the obtained ethylene glycol composition is shown in Table 3.

TABLE 3
Ethylene glycol composition table

| | 6 | | | |
|---|---|---|---|---|
| | Ethylene glycol | | 8 | |
| | solution containing a | | Final | |
| | high concentration | | ethylene glycol | |
| Content | of specific alcohol | | composition | |
| ethylene glycol | 72.8 | wt % | 99.7 | wt % |
| 1,2-propanediol + | 3 | wt % | 198 | ppm |
| 2-methyl-1,3-propanediol | | | | |
| 1,2-butanediol | 22.6 | wt % | 1506 | ppm |
| 1,2-, 1,3-, 1,4-, 1,5-hexanediol | 1.6 | wt % | 404 | ppm |

### Example 4

Example 1 is repeated, except that the distillation conditions of the specific alcohol recovery tower are changed. The specific distillation conditions are as follows: theoretical plate number 50, tower top temperature 216° C., tower bottom temperature 250° C., and normal pressure operation. 6 and 5 are mixed at a weight mixing ratio of 1:150. Data of the obtained ethylene glycol composition is shown in Table 4.

TABLE 4
Ethylene glycol composition table

| | 6 | | | |
|---|---|---|---|---|
| | Ethylene glycol | | 8 | |
| | solution containing a | | Final | |
| | high concentration | | ethylene glycol | |
| Content | of specific alcohol | | composition | |
| ethylene glycol | 70.2 | wt % | 99.7 | wt % |
| 1,2-propanediol + | 4 | wt % | 264 | ppm |
| 2-methyl-1,3-propanediol | | | | |
| 1,2-butanediol | 23.6 | wt % | 1577 | ppm |
| 1,2-, 1,3-, 1,4-, 1,5-hexanediol | 3.1 | wt % | 505 | ppm |

### Example 5

Example 1 is repeated, except that the distillation conditions of the specific alcohol recovery tower are changed. The specific distillation conditions are as follows: theoretical plate number 50, tower top temperature 218° C., tower bottom temperature 249° C., and normal pressure operation. 6 and 5 are mixed at a weight mixing ratio of 1:150. Data of the obtained ethylene glycol composition is shown in Table 5.

TABLE 5
Ethylene glycol composition table

| | 6 | | | |
|---|---|---|---|---|
| | Ethylene glycol | | 8 | |
| | solution containing a | | Final | |
| | high concentration | | ethylene glycol | |
| Content | of specific alcohol | | composition | |
| ethylene glycol | 70.9 | wt % | 99.7 | wt % |
| 1,2-propanediol + | 2.8 | wt % | 186 | ppm |
| 2-methyl-1,3-propanediol | | | | |
| 1,2-butanediol | 24.2 | wt % | 1611 | ppm |
| 1,2-, 1,3-, 1,4-, 1,5-hexanediol | 2.1 | wt % | 437 | ppm |

### Performance Test

The ethylene glycol compositions obtained from Examples 1-5 and Comparative Example 1 are respectively reacted with terephthalic acid at a molar ratio of 1:1 in the presence of 0.5% stannous octoate catalyst based on the total weight of the monomers at a temperature of 180-260° C. and a pressure of 0.2-0.4 MPa for esterification for 3 hours, and then polycondensed at a temperature of 220-290° C. and a pressure of 10-60 Pa to obtain a polyester. The intrinsic viscosity, tensile strength, elongation at break, glass transition temperature (Tg) and other properties are tested respectively.

The polyester is extruded into granules and placed in a vacuum dryer for drying. The temperature of the first stage is controlled at 80° C. for 2 hours, the temperature of the second stage is controlled at 110° C. for 1 hour, and the temperature of the third stage is controlled at 120° C., and the drying time is controlled to be about 24 hours. Then, a general spinning procedure is used to melt it at 260° C., and melt spinning is performed. The obtained polyester fiber is subjected to a cationic low-temperature dyeing test.

### Dyeing Conditions

Dye: Cathilon Red CD-FGLH 3.0% omf
Auxiliary agents: Na₂SO₄ 10%, CH₃COONa 0.5%, CH₃COOH (50%)
Bath ratio: 1:50
Dyeing temperature×time: 90° C.×40 minutes

### Dyeing Rate:

The original solution before dyeing and the residual solution after dyeing are diluted with acetone aqueous solution (acetone: water=1:1). The absorbance is then measured, and the dyeing rate is calculated using the following formula.

$$Dyeing rate = ( A - B ) / A \times 100 \%$$

A: Absorbance of original solution (acetone aqueous solution dilution)
B: Absorbance of dyed residual solution (acetone aqueous solution dilution)

A dyeing rate of more than 90% is qualified, recorded as 0; a dyeing rate of less than 90% is unqualified, recorded as x.

The dyeing results are shown in Table 6.

TABLE 6

| | Intrinsic | Tensile | Melting | | Elongation | Low- |
|---|---|---|---|---|---|---|
| | viscosity | strength | point | Tg | at break | temperature |
| | (g/dL) | (MPa) | Tm (° C.) | (° C.) | (%) | dyeability |
| Example 1 | 0.65 | 52 | 243 | 58 | 270 | ∘ |
| Example 2 | 0.644 | 53.4 | 246 | 56 | 280 | ∘ |
| Example 3 | 0.636 | 55.2 | 242 | 53 | 300 | ∘ |
| Example 4 | 0.641 | 52.1 | 247 | 54 | 290 | ∘ |
| Example 5 | 0.646 | 53.7 | 249 | 57 | 275 | ∘ |
| Comparative | 0.61 | 49 | 241 | 69 | 110 | x |
| Example 1 | | | | | | |

As shown in Table 6, compared with Comparative Example 1, the polyester fiber prepared from the ethylene glycol composition of the present invention has comparable mechanical properties to those of the prior art and better low-temperature dyeability.

What is claimed is:

1. An ethylene glycol composition, based on a total weight of the ethylene glycol composition, comprising
(1) at least 99.0% ethylene glycol,
(2) 110-5000 ppm propylene glycol,
(3) 110-10000 ppm butanediol, and
(4) 110-10000 ppm hexanediol.

2. The ethylene glycol composition according to claim 1, wherein the propylene glycol is 1,2-propylene glycol, 1,3-propylene glycol, or 2-methyl-1,3-propylene glycol.

3. The ethylene glycol composition according to claim 1, wherein the butanediol is 1,2-butanediol, 1,3-butanediol, or 1,4-butanediol.

4. The ethylene glycol composition according to claim 1, wherein the hexanediol is 1,2-hexanediol, 1,3-hexanediol, 1,4-hexanediol, 1,5-hexanediol, or 1,6-hexanediol.

5. The ethylene glycol composition according to claim 1, based on the total weight of the ethylene glycol composition, comprising:
(1) at least 99.0% the ethylene glycol,
(2) 110-5000 ppm 1,2-propylene glycol or 2-methyl-1,3-propylene glycol,
(3) 110-10000 ppm 1,2-butanediol or 1,3-butanediol, and
(4) 110-10000 ppm 1,2-hexanediol, 1,3-hexanediol, 1,4-hexanediol or 1,5-hexanediol.

6. A method for preparing an ethylene glycol composition, comprising steps of:
(1) reacting CO with methanol to generate dimethyl oxalate, hydrogenating the dimethyl oxalate to generate ethylene glycol, and passing the ethylene glycol through a methanol recovery tower and a dehydration tower to obtain a crude ethylene glycol;
(2) feeding the crude ethylene glycol to a light fraction removal tower, feeding a heavy fraction of the light fraction removal tower to an ethylene glycol product tower, and distilling the heavy fraction of the light fraction removal tower in the ethylene glycol product tower to obtain an ethylene glycol product;
(3) feeding a light fraction of the light fraction removal tower to a specific alcohol recovery tower, distilling the light fraction of the light fraction removal tower in the specific alcohol recovery tower, and obtaining an ethylene glycol solution containing a high concentration of a specific alcohol from a bottom of the specific alcohol recovery tower; and
(4) mixing the ethylene glycol solution containing the high concentration of the specific alcohol obtained in the step (3) with the ethylene glycol product obtained in the step (2) in appropriate amounts according to a desired concentration of the ethylene glycol composition.

7. A polyester, comprising a polymerized monomer unit derived from the ethylene glycol composition according to claim 1.

8. The polyester according to claim 7, further comprising a polymerized monomer unit derived from one or more of terephthalic acid, phthalic acid, isophthalic acid, and naphthalene dicarboxylic acid.

9. A method for producing polyester resins, antifreeze solutions, or unsaturated resins, comprising using the ethylene glycol composition according to claim 1.

10. A fiber, comprising the polyester according to claim 7.

11. The ethylene glycol composition according to claim 2, wherein the butanediol is 1,2-butanediol, 1,3-butanediol, or 1,4-butanediol.

12. The ethylene glycol composition according to claim 2, wherein the hexanediol is 1,2-hexanediol, 1,3-hexanediol, 1,4-hexanediol, 1,5-hexanediol, or 1,6-hexanediol.

13. The ethylene glycol composition according to claim 3, wherein the hexanediol is 1,2-hexanediol, 1,3-hexanediol, 1,4-hexanediol, 1,5-hexanediol, or 1,6-hexanediol.

14. The polyester according to claim 7, wherein in the ethylene glycol composition, the propylene glycol is 1,2-propylene glycol, 1,3-propylene glycol, or 2-methyl-1,3-propylene glycol.

15. The polyester according to claim 7, wherein in the ethylene glycol composition, the butanediol is 1,2-butanediol, 1,3-butanediol, or 1,4-butanediol.

16. The polyester according to claim 7, wherein in the ethylene glycol composition, the hexanediol is 1,2-hexanediol, 1,3-hexanediol, 1,4-hexanediol, 1,5-hexanediol, or 1,6-hexanediol.

17. The polyester according to claim 7, wherein the ethylene glycol composition, based on the total weight of the ethylene glycol composition, comprises:
(1) at least 99.0% the ethylene glycol,
(2) 110-5000 ppm 1,2-propylene glycol or 2-methyl-1,3-propylene glycol,
(3) 110-10000 ppm 1,2-butanediol or 1,3-butanediol, and
(4) 110-10000 ppm 1,2-hexanediol, 1,3-hexanediol, 1,4-hexanediol, or 1,5-hexanediol.

18. The method according to claim 9, wherein in the ethylene glycol composition, the propylene glycol is 1,2-propylene glycol, 1,3-propylene glycol, or 2-methyl-1,3-propylene glycol.

19. The method according to claim 9, wherein in the ethylene glycol composition, the butanediol is 1,2-butanediol, 1,3-butanediol, or 1,4-butanediol.

20. The method according to claim 9, wherein in the ethylene glycol composition, the hexanediol is 1,2-hexanediol, 1,3-hexanediol, 1,4-hexanediol, 1,5-hexanediol, or 1,6-hexanediol.
